(19) United States
(12) Patent Application Publication
(10) Pub. No.: US 2003/0157063 A1
(43) Pub. Date: Aug. 21, 2003

(54) Gene therapy using anti-gp41 antibody and CD4 immunoadhesin

(76) Inventors: Jean-Louis Touraine, Lyon (FR); Kamel Sanhadji, Lyon (FR); Pierre Leroy, Ernolsheim Les Saverne (FR); Majid Mehtali, Plobsheim (FR)

Correspondence Address: OLIFF & BERRIDGE, PLC, P.O. BOX 19928, ALEXANDRIA, VA, 22320, US

(21) Appl. No.: 10/024,329

(22) Filed: Dec. 21, 2001

(51) Int. Cl. A61K 48/00; C12N 5/08

(52) U.S. Cl. 424/93.2; 514/44; 424/93.21; 435/372

(57) ABSTRACT

A gene therapy composition of the present invention comprises one or more nucleotide fragments. The one or more nucleotide fragments taken together comprise at least (1) a nucleotide sequence (a) encoding human soluble CD4, (2) a nucleotide sequence (b) comprising at least nucleotide sequences encoding the heavy chain and the light chain of immunoglobulin IgG3, wherein the IgG3 is directed against at least one of the peptides selected from the group consisting of SEQ ID NO:2 to SEQ ID NO:26. The composition further comprises the nucleic elements required for replicating each of nucleotide sequences (a) and (b), in a host cell, when the host cell divides and for expressing under control each of nucleotide sequences (a) and (b) in the host cell.

This invention discloses a method for treating infectious diseases, in particular viral HIV-1 infection, by gene therapy, and the genetic recombinant means to implement this treatment.

One means of combating HIV-1 infection would be to provide seropositive patients with passive immunoserotherapy, using soluble molecules directed against the virus, most particularly viral proteins, to obtain the neutralization of HIV in vivo [10]. Experiments involving the inoculation of virus into monkeys, together with the administration of anti-HIV-1 immunoglobulins, demonstrated the feasibility of the prevention of infection [11,13]. Histopathological, immunological and virological characteristics in the protected animals were strikingly similar to those observed in long-term human survivors with non-progressive HIV-1 infection [14].

An anti-gp41 monoclonal antibody (2F5mAb) [36] directed against the envelope glycoprotein gp41 of HIV-1 displayed a potent neutralization effect in vitro and in vivo, either alone or in various combinations with other monoclonal antibodies or with hyperimmune globulins [15,13,16,17].

The neutralization of HIV-1 mediated by a soluble form of CD4 (sCD4) which is the primary receptor for viral attachment to the envelope glycoprotein gp120 has also been demonstrated, either alone [18,19,49] or in combination with V3 loop monoclonal antibodies [20]. sCD4 acts as a decoy towards HIV-1 gp120 thus limiting the binding of this retroviral glycoprotein onto the CD4 receptor of T-cells, which is the key for entering and infecting the cells.

WO-94/19017 [49] discloses a pharmaceutical composition comprising sCD4, 2F5 monoclonal antibody and a carrier, which is administered to an HIV-1-infected patient, in an amount effective to reduce the rate of spread of the HIV-1 and infection.

Moreover, in each of the above-mentioned prior art therapeutic solutions, the treatment induces in the patient a growing immunological response directed against the therapeutic molecule. Consequently, the necessary quantities of the serotherapeutic agent rapidly increase as the treatment goes on, up to quantities that are no longer compatible for human administration, since they may amount to huge volumes after several weeks of treatment. In this respect, passive immunoserotherapy suffered limitation as a treatment option, and has thus been abandoned.

It has been evidenced that delivering sCD4, by gene therapy, provided an efficient inhibition of HIV-1 infection in vitro [23]. Morgan RA et al. have actually reported that in a co-culture of either human T-cell lines or primary T-cells with sCD4-producing NIH 3T3 cells, the T-cell lines or T-cells obtained by the coculture were protected after HIV-1 challenge.

This therapy which may also apply to HIV-2 infection as the envelope glycoprotein gp105 of HIV-2 binds to CD4 receptor, is nevertheless insufficient and may not be safe, because, firstly, in case the gene encoding the said sCD4 is not expressed in the host cell, no therapeutic effect will affect the patient, and secondly, even if the binding site of the glycoprotein gp120 with said CD4 receptor is rather constant from one HIV-1 isolate to another, the least mutation in this region would result in a total failure of the treatment. It should be reminded that HIV-1 is naturally a highly variable retrovirus, and that the AIDS dual- and triple-therapy based on antiviral drugs has greatly promoted the generation of mutants.

There is still a great need for an efficient therapy that could apply to most HIV-1 strains.

According to the present invention, the anti-gp41 2F5mAb and a sCD4-based molecule were used for the first time in an efficient dual gene therapy.

The inventors have now discovered first that the production of both 2F5 monoclonal antibody and said sCD4-based molecule can be maintained continuously in vivo, and secondly that this production leads to a stable neutralization of HIV-1 by persistently and efficiently reducing the HIV-1 load in vivo.

The present invention provides a composition comprising one or more a nucleotide fragments, wherein the one or more nucleotide fragments taken together comprise at least (1) an nucleotide sequence (a) encoding human soluble CD4, (2) an nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and (3) the nucleic elements required for replicating each said nucleotide sequences (a) and (b), in a host cell, when said host cell divides and for expressing under control each of said nucleotide sequences (a) and (b) in said host cell.

The composition according to the invention may comprise:

an expression cassette comprising at least nucleotide sequence (a) and nucleotide sequence (b) and the nucleic elements required for expressing them under control in said host cell;

a first expression cassette comprising nucleotide sequence (a), and the nucleic elements required for expressing it under control in said host cell, and a second expression cassette comprising nucleotide sequence (b), and the nucleic elements required for expressing it under control in said host cell;

a vector comprising at least nucleotide sequence (a) and nucleotide sequence (b), and the nucleic elements required for expressing them under control in said host cell;

at least, a first recombinant vector comprising nucleotide sequence (a), and the nucleic elements required for expressing it under control in said host cell, and a second recombinant vector comprising nucleotide sequence (b), and the nucleic elements required for expressing it under control in said host cell.

In order to obtain constitutive secretion, that is a continuous expression, of 2F5mAb or of a monoclonal antibody directed against the same epitope as the one against which 2F5mAb is directed, said epitope consisting of an antigenic nucleotide sequence selected among the group consisting of SEQ ID NO:2 to SEQ ID NO:26, and of sCD4, in vivo, the inventors have first developed cell lines genetically modified by the vectors of the invention, and which have then been incorporated in collagen fibers or synthetic tissues to form neo-organs that could be grafted intraperitoneally in SCID mice.

Severe combined immune deficient (SCID) mice which are acknowledged animal models, have been used as recipients for human cell grafts [6,7]. Such humanized SCID mice were then shown to be readily infectable with HIV [8], and this model was recently used to test experimental gene therapy delivering interferons against HIV-1 infection [9].

As it will be illustrated in the examples, three types of neo-organs (2F5, sCD4-IgG, and 2F5 +sCD4-IgG) were generated to provide long-term delivery of recombinant molecules in vivo. These neo-organs became strongly vascularized within a few weeks of their implantation; they were not rejected and ensured secretion of the desired molecules into the blood stream of the animals. This experimental model has been evidenced remarkable by the inventors for short term observations.

According to the invention, the neo-organs have been obtained from NIH3T3 murine fibroblasts genetically modified in vitro, by transduction with a recombinant vector of the invention. Their implantation into SCID mice led the continuous production of 2F5 which could be obtained for up to 6 weeks at serum concentrations close to 1 μg/ml.

Similarly, a soluble form of the CD4 molecule has been shown to block the interaction of CD4 cell with gp120, thus inhibiting HIV infection [37,38] as mentioned above. It was further demonstrated that the effect was dose-dependent when sCD4 cells were maintained throughout cell culture [39]. The continuous secretion of sCD4 by somatic transgenesis ensured their presence for 2 months after a single transgenesis in mice, whereas administration in vivo resulted in the short-term presence of the molecule, for a few hours [40]. Therefore, the inventors used a second-generation CD4-based molecule (named sCD4-IgG) which involves the genetic coupling of a portion of the CD4 structure (sCD4) to the Fc fragment of an IgG molecule. Such stabilized immunoglobulins or immunoadhesins [41] displayed the same affinity as sCD4 for gp120 and have a longer half-life in vivo [42].

In previous pilot experiments, the inventors confirmed the antiviral efficacy of the 2F5 recombinant molecules. Using 2F5 neo-organs, they observed that the intensity of the antiviral effect was dependent on the dose of antibody produced in vivo. It was found that plasma levels of 2F5 approximating 1 μg/ml in SCID mice were required to induce a regular and significant reduction in the virus load. In the present experiments, supporting the present invention, with the endogeneous production of 2F5 after neo-organ implantation, not only was the number of RNA copies of HIV-1 in SCID-CEM mice very significantly decreased, but also the cellular viral load and reverse transcriptase activity were reduced.

Furthermore, when produced by neo-organs in vivo, the sCD4-IgG molecule is effective in significantly reducing the viral load of HV-infected SCID-CEM mice.

Either 2F5 or sCD4-IgG can inhibit HIV infection in vivo. Their potential antiviral efficiency was directly related to their ability to interact with the HIV-1 envelope. This would affect HIV-1 propagation by preventing the virus from infecting its target cells because of their competitive or neutralizing properties.

It has been discovered that when both 2F5 and sCD4-IgG are produced together, concomitantly or more or less concomitantly, a cooperation or complementary effect between 2F5 and sCD4 occurs in vivo. This effect which is evidenced in examples provides a safety degree of the treatment much higher than the sCD4 single agent therapy, above-mentioned, in that the treatment is effective irrespectively of the mutations or variability of the infecting virus, and reproducible from one mammal or patient to another one.

It is believed that the binding of sCD4-IgG on gp120 would facilitate the binding of 2F5 on gp41, by inducing a conformational change of the envelope and therefore increases the action of neutralizing 2F5 antibodies.

This complementary effect could result from the following mechanism: gp120 is a large and readily accessible glycoprotein which hinders the access to gp41; gp41 is a smaller glycoprotein and is “fitted” onto gp120. The binding of sCD4-IgG to the gp120 modifies the conformation of gp120, freeing the epitope region of gp41. As this access is made easier, 2F5 antibodies readily and efficiently bind to gp41, enhancing consequently the neutralizing activity of the combination sCD4-IgG and 2F5 compared to the simple additive effect of sCD4-IgG and 2F5.

Definition:

Human immunodeficiency virus type 1 (HIV-1) is the principal etiologic agent of AIDS as described by Barré-Sinoussi F et al. [51], Gallo RC et al. [52], and which nucleotide sequence has been reported by Wain-Hobson S et al. [53] and Ratner L et al. [54].

Soluble CD4 (sCD4) is an extracellular part of the human CD4 glycoprotein receptor of T-cells interacting with HIV-1 [55], said extracellular part of CD4 consisting of four variable domains, D1, D2, D3 and D4, bound by joining domains, J1, J2, J3 and J4, respectively and of a leader sequence L, and may represented for example by SEQ ID NO:32. According to the present invention, sCD4 is a polypeptide comprising at least L, D1, J1 and D2 domains. Preferably, sCD4 is a recombinant peptide illustrated by SEQ ID NO:33, encoded by SEQ ID NO:31. sCD4 is soluble in an aqueous solution including detergent-free aqueous buffer and body fluids such as blood, plasma and serum.

sCD4 multimer is a recombinant multimer protein comprising at least four segments each consisting of L, D1, J1 and D2 domains of CD4 (refer to WO-97/04109). sCD4-IgG is a fusion protein which results in the fusion of sCD4 and the constant region of an immunoglobulin, for example 2F5mAb which preparation is described in WO-96/08574.

2F5 [36] is a human monoclonal antibody directed to the epitope sequence SEQ ID NO:2 belonging to the external domain of the gp41 envelope glycoprotein of most HIV-1 strains, or to an immunological equivalent sequence selected from the group consisting of SEQ ID NO:3-SEQ ID NO:26 [56,57]. 2F5mAb may be purchased from Virus Testing Systems (Houston, USA) and hybridoma cell line producing said mAb may be prepared from peripheral blood mononuclear cells from HIV-1-infected patients which are then fused, and selected as described by Buchacher M. et al. [36].

The nucleotide sequences encoding respectively the light and the heavy chains of 2F5 may be obtained as follows:

The complementary DNA (cDNA) encoding the light chain is included in HindIII-EcoRI fragment in vector Bluescript SK+(purchased from Stratagene) . This cDNA has been obtained from a cDNA library originating from the mRNA of hybridoma 2F5 [28] by using primers identified by SEQ ID NO:27 and 28.

The complementary DNA (cDNA) encoding the heavy chain is included in NcoI-EcoRI fragment in vector pTG2677 described in FIG. 1. This cDNA has been obtained from a cDNA library originating from the mRNA of hybridoma 2F5 [28] by using primers identified by SEQ ID NO:29 and 30.

Nucleic elements contained in a composition, in an expression cassette or in a vector of the invention, and which is required for expressing specified sequences, under control, in a host cell, may be exogenous or endogenous elements; this expression is carried out under control, that is the control of at least one step of the expression process, selected among the group consisting of transcription, maturation of RNA, transport of RNA, translation, degradation.

A neo-organ, also designated as organoid or implant, is an organ which has been obtained in laboratory, starting from human or animal cells that are cultivated in vitro on an extra-cellular polymer matrix. When the cells have reached the desired growth level, the neo-organ is implanted in a human or animal. According to the invention, said implant comprises living modified cells which are able to produce at least one molecule of interest. The neo-organ thus implanted is capable of continuously releasing in the recipient, in vivo, said molecule of interest. The matrix is preferably made of at least one bio-compatible material selected from collagen, polytetrafluoroethylene, Gore-Tex™ [75]

The nucleotide sequences (a) and (b) to which the present invention pertains can be cDNA or genomic sequences or be of a mixed type. It can, where appropriate, contain one or more introns, with these being of native, heterologous (for example the intron of the rabbit β-globin gene, etc.) or synthetic origin, in order to increase expression in the host cells.

The nucleotide sequences employed within the context of the present invention can be obtained by the conventional techniques of molecular biology, for example by screening libraries with specific probes, by immunoscreening expression libraries or by PCR using suitable primers, or by chemical synthesis.

As previously mentioned, the present invention also relates to at least one recombinant vector, or two recombinant vectors which comprise nucleotide sequences (a) and (b) according to the invention, which is placed under the control of the nucleic elements which are required for expressing it in a host cell.

A vector according to the present invention, or nucleic acid construct, devoted to gene therapy, may be used in its naked form [58], combined with liposomes, cationic lipids, cationic polymers, peptides or polypeptides. The literature relating to the vectors that may be used in gene therapy provides a considerable numbers of examples of such vectors [see for example 59].

The recombinant vectors can be of plasmid or viral origin and can, where appropriate, be combined with one or more substances which improve the transfectional efficiency and/or stability of the vectors. These substances are widely documented in the literature which is available to the skilled person (see, for example, 60, 61, 62]. By way of non-limiting illustration, the substances can be polymers, lipids, in particular cationic lipids, liposomes, nuclear proteins or neutral lipids. These substances can be used alone or in combination. A combination which can be envisaged is that of a recombinant plasmid vector which is combined with cationic lipids (DOGS, DC-CHOL, spermine-chol, spermidine-chol, etc.) and neutral lipids (DOPE).

The choice of the plasmids which can be used within the context of the present invention is immense. They can be cloning vectors and/or expression vectors. In a general manner, they are known to the skilled person and, while a number of them are available commercially, it is also possible to construct them or to modify them using the techniques of genetic manipulation. Examples which may be mentioned are the plasmids which are derived from pBR322 (Gibco BRL), pUC (Gibco BRL), pBluescript (Stratagene), pREP4, pCEP4 (Invitrogene) or p Poly [63]. Preferably, a plasmid which is used in the context of the present invention contains an origin of replication which ensures that replication is initiated in a producer cell and/or a host cell (for example, the oriP/EBNA1 system will be chosen if it's desired that the plasmid should be self-replicating in a mammalian host cell [64,65]). The plasmid can additionally comprise a selection gene which enables the transfected cells to be selected or identified (complementation of an auxotrophic mutation, gene encoding resistance to an antibiotic, etc.). Certainly, the plasmid can contain additional elements which improve its maintenance and/or its stability in a given cell (cer sequence) which promotes maintenance of a plasmid in monomeric form [66].

The recombinant vectors may be independently selected from adenoviral vectors, lentiviral vectors [67], “new generation” adenoviral vectors, retroviral vectors, vectors which are derived from a poxvirus (vaccinia virus, in particular MVA, canarypoxvirus, etc.), vectors which are derived from a herpesvirus, from an alphavirus, from a foamy virus or from an adenovirus-associated virus, chimeric viral vectors and synthetic vectors [68].

Retroviruses have the property of infecting, and in most cases integrating into, dividing cells and in this regard are particularly appropriate for use in relation to cancer. A recombinant retroviral vector according to the invention generally contains the LTR sequences, an encapsidation region and at least one of the nucleotide sequence according to the invention, which is placed under the control of the retroviral LTR or of an internal promoter such as those described below. The recombinant retroviral vector can be derived from a retrovirus of any origin (murine, primate, feline, human, etc.) and in particular from the MoMuLV (Moloney murine leukemia virus), MVS (Murine sarcoma virus) or Friend murine retrovirus (Fb29). It is propagated in an encapsidation cell line which is able to supply in trans the viral polypeptides gag, pol and/or env which are required for constituting a viral particle. Such cell lines are described in the literature (PA317, Psi CRIP GP+Am-12 etc.). The retroviral vector according to the invention can contain modifications, in particular in the LTRs (replacement of the promoter region with a eukaryotic promoter) or the encapsidation region (replacement with a heterologous encapsidation region, for example the VL30 type).

Preference will be given to using a vector which does not replicate and does not integrate. In this respect, adenoviral vectors are very particularly suitable for implementing the present invention.

Accordingly, the prefered use is of an adenoviral vector which lacks all or part of at least one region which is essential for replication and which is selected from the E1, E2, E4 and L1-L5 regions in order to avoid the vector being propagated within the host organism or the environment. A deletion of the E1 region is preferred. However, it can be combined with (an)other modification(s)/deletion(s) affecting, in particular, all or part of the E2, E4 and/or L1-L5 regions, to the extent that the defective essential functions are complemented in trans by means of a complementing cell line and/or a helper virus. In this respect, it is possible to use “new generation” vectors of the state of the art. By way of illustration, deletion of the major part of the E1 region and of the E4 transcription unit is very particularly advantageous. For the purpose of increasing the cloning capacities, the adenoviral vector can additionally lack all or part of the non-essential E3 region. According to another alternative, it is possible to make use of a minimal adenoviral vector which retains the sequences which are essential for encapsidation, namely the 5′ and 3′ ITRs (Inverted Terminal Repeat), and the encapsidation region. The various adenoviral vectors, and the techniques for preparing them, are known [see, for example 69].

Furthermore, the origin of the adenoviral vector according to the invention can vary both from the point of view of the species and from the point of view of the serotype. The vector can be derived from the genome of an adenovirus of human or animal (canine, avian, bovine, murine, ovine, porcine, simian, etc.) origin or from a hybrid which comprises adenoviral genome fragments of at least two different origins. More particular mention may be made of the CAV-1 or CAV-2 adenoviruses of canine origin, of the DAV adenovirus of avian origin or of the Bad type 3 adenovirus of bovine origin [70,71,72,73]. However, preference will be given to an adenoviral vector of human origin which is preferably derived from a serotype C adenovirus, in particular a type 2 or 5 serotype C adenovirus.

An adenoviral vector according to the present invention can be generated in vitro in Escherichia coli (E. coli) by ligation or homologous recombination or else by recombination in a complementing cell line.

The nucleic elements required for expression consist of all the elements which enable the nucleotide sequence to be transcribed into RNA and the mRNA to be translated into polypeptide. These elements comprise, in particular, a promoter which may be regulatable or constitutive. Logically, the promoter is suited to the chosen vector and the host cell. Examples which may be mentioned are the eukaryotic promoters of the PGK (phosphoglycerate kinase), MT (metallothionein) [84], α-1 antitrypsin, CFTR, surfactant, immunoglobulin, β-actin [76] and SRα [77] genes, the early promoter of the SV40 virus (Simian virus), the LTR of RSV (Rous sarcoma virus), the HSV-1 TK promoter, the early promoter of the CMV virus (Cytomegalovirus), the p7.5K pH5R, pK1L, p28 and p11 promoters of the vaccinia virus, and the E1A and MLP adenoviral promoters. The promoter can also be a promoter which stimulates expression in a tumor or cancer cell. Particular mention may be made of the promoters of the MUC-l gene, which is overexpressed in breast and prostate cancers [78], of the CEA (standing for carcinoma embryonic antigen) gene, which is overexpressed in colon cancers [79] of the tyrosinase gene, which is overexpressed in melanomas [80], of the ERBB-2 gene, which is overexpressed in breast and pancreatic cancers [81] and of the α-fetoprotein gene, which is overexpressed in liver cancers [82]. The cytomegalovirus (CMV) early promoter is very particularly preferred.

The necessary elements can furthermore include additional elements which improve the expression of the nucleotide sequences (a) and/or (b) according to the invention or its maintenance in the host cell. Intron sequences, secretion signal sequences, nuclear localization sequences, internal sites for the reinitiation of translation of IRES type, transcription termination poly A sequences, tripartite leaders and origins of replication may in particular be mentioned. These elements are known to the skilled person.

The invention also relates to:

a host cell which comprises at least an expression cassette of the invention as previously described;

a host cell which comprises at least a vector of the invention as previously described;

wherein said host cell may be selected among the group consisting of fibroblasts, lymphocytes and stem cells;

a tissue of genetically modified cells comprising a plurality of host cells previously described.

an implant of genetically modified cells comprising a plurality of host cells previously described.

said tissue or implant wherein said host cells are selected among the group consisting of fibroblasts, lymphocytes, stem cells.

The invention also relates to a method for treating an infectious disease, wherein the following medication may be administered by gene therapy, to a mammal or a patient, in particular when said infectious disease is caused by HIV-1 retrovirus:

a composition of the invention as previously described

at least (1) a first expression cassette comprising a nucleotide sequence (a) encoding human soluble CD4 and nucleic elements required for replicating nucleotide sequence (a) in a host cell, when said host cell divides, and for expressing under control said nucleotide sequence (a) in said host cell, (2) a second expression cassette comprising a nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and nucleic elements required for replicating nucleotide sequence (b), when said host cell divides, and for expressing under control said nucleotide sequence (b) in said host cell; said first expression cassette and said second expression cassette may be administered, concomitantly or separately;

at least (1) a first recombinant vector comprising a nucleotide sequence (a) encoding human soluble CD4 and nucleic elements required for replicating nucleotide sequence (a) in a host cell, when said host cell divides, and for expressing under control said nucleotide sequence (a) in said host cell, (2) a second recombinant vector comprising a nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and nucleic elements required for replicating nucleotide sequence (b), when said host cell divides, and for expressing under control said nucleotide sequence (b) in said host cell; said first recombinant vector and said second recombinant vector may be administered, concomitantly or separately;

at a least one host cell of the invention as previously described;

an implant of the invention as previously described.

A composition according to the invention can be made conventionally with a view to administering it locally, parenterally or by the digestive route. In particular, a therapeutically effective quantity of the therapeutic or prophylactic agent is combined with a pharmaceutically acceptable excipient. It is possible to envisage a large number of routes of administration. Examples which may be mentioned are the intramuscular, intratracheal, intratumoral, intragastric, intraperitoneal, epidermal, intracardiac, intraperitoneal, intravenous or intraarterial route, by inhalation, by instillation, by aerosolization, by the topical route or by the oral route. In the case of these three latter embodiments, it is advantageous for administration to take place by means of an aerosol or by means of instillation. The administration can take place as a single dose or as a dose which is repeated on one or more occasions after a particular time interval. The appropriate route of administration and dosage vary depending on a variety of parameters, for example the individual to be treated, the vector which has been selected for the gene therapy. For example, the composition according to the invention can be formulated in the form of doses of about 890 ng/ml for 2F and 77 ng/ml for sCD4-IgG if the vector is of retroviral type, and in the form of doses of about 1 mg/ml for each of 2F5 and sCD4-IgG is the vector is of adenoviral type. Naturally, the doses can be adjusted by the clinician.

The composition can also include a diluent, an adjuvant or an excipient which is acceptable from the pharmaceutical point of view, as well as solubilizing, stabilizing and preserving agents. In the case of an injectable administration, preference is given to a formulation in an aqueous, non-aqueous or isotonic solution. It can be presented as a single dose or as a multidose, in liquid or dry (powder, lyophilizate, etc.) form which can be reconstituted at the time of use using an appropriate diluent.

The composition of the invention can be administered directly in vivo (for example by intravenous injection, into the lungs by means of an aerosol, into the vascular system using an appropriate catheter, etc.). It is also possible to adopt the ex vivo approach, which consists in removing cells from the patient (stem cells, peripheral blood lymphocytes, etc.), transfecting or infecting them in vitro in accordance with the techniques of the art and then readministering them to the patient.

### FIGURES

FIG. 1 is a schematic representation of the retroviral vector RVTG6371; RVTG6371 encodes the human monoclonal antibody (mAb) 2F5.

It carries the long terminal repeat of Moloney murine sarcoma virus (5′LTR), the Moloney murine sarcoma virus/Moloney murine leukemia virus hybrid packaging sequences (psi), the mouse phosphoglycerate kinase-1 gene promoter sequences (PGK), the mAb 2F5 heavy and light chain cDNA, (respectively 2F5 HC and 2F5 LC), the internal ribosome entry site (IRES) of encephalomyocarditis virus, the long terminal repeat (3′LTR) of myeloproliferative sarcoma virus, the puromycine acetyltransferase (Pac) gene and the simian virus polyadenylation (PA) signal.

FIG. 2 is a schematic representation of the retroviral vector RVTG8338; RVTG8338 encodes the human soluble CD4-IgG immunoadhesin (sCD4-IgG).

Its construction is based on the ligation of the leader variable (V1/V2) segment of human CD4 (SEQ ID NO:30), 2F5 hinge region (Hinge CH₂-CH₃) and the 2F5 heavy chain (2F5 HC) sequence.

FIG. 3 illustrates the in vivo secretion of 2F5 monoclonal antibody:NIH3T3TG6371 cells were implanted intraperitoneally into SCID mice; the presence of the 2F5 mAb, in animal sera was detected and followed for 2 months using enzyme-linked immunosorbent assay.

FIG. 4 is a schematic representation of the structure of CD4 receptor.

FIG. 5 is a schematic representation of a vector encoding the multimer protein sCD4.

FIG. 6 represents the viral load in SCID-hu-HIV-1-sCD4-IgG-2F5 mice ×1000 (mRNA copies/ml) versus time in days.

### EXAMPLES

Examples of the present invention are also described in Sanhadji K. et al. [83], which is herein incorporated in its entirety by reference.

### Example 1

### Construction of Vectors

The complementary DNA encoding the heavy chain (HC) and light chain (LC) of 2F5mAb have been obtained as indicated above, in the definition part of the description.

The sequence of sCD4-IgG results from the ligation of the segment L-D1-J1-D2 (SEQ ID NO:31) of human CD4 with the constant region of 2F5.

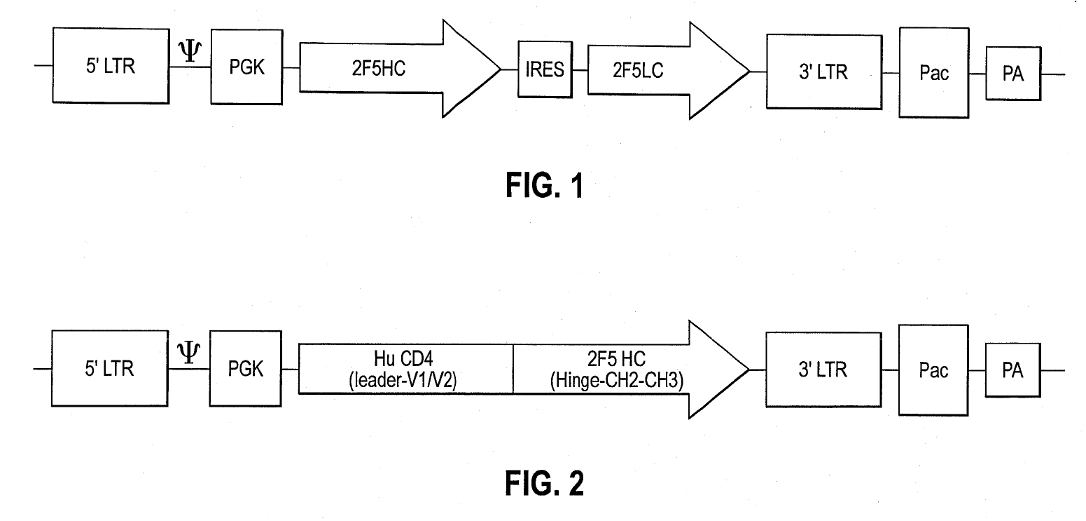

Both were inserted into retroviral vectors from Moloney murine leukemia virus, noted respectively RVTG6371 (illustrated on FIG. 1) and RVTG8338 (illustrated on FIG. 2). To avoid the gradual inactivation of retrovirally transferred expression cassette in vivo, the promoter of mouse phosphoglycerate kinase type I was used. High titres of transgene products were obtained with these constructions and extinction has never been observed in vitro.

The dicistronic vector RVTG6371, was used to give a sub-equivalent quantity of heavy chain sequence and light chain sequence of 2F5mAb directed against the ELDKWAS linear epitope (SEQ ID NO:2) of HIV-1 gp41.

The amount of sCD4-IgG obtained from RVTG8338 could not be quantified, but the molecules were detected by immunofluorescence, enzyme-linked immunosorbent assay (ELISA) and Western blot techniques.

### Example 2

### Construction of Modified Cell Lines

NIH-3T3 fibroblasts were stably transduced with RVTG6371 or RVTG8338 encoding respectively 2F5mAb and sCD4-IgG, to obtain respectively NIH-3T3TG6371 and NIH-3T3TG8338. The resulting cells were shown constitutively to produce the 2F5mAb or the sCD4-IgG immunoadhesin through the analysis of culture supernatants by a Western blot assay and ELISA and by a specific immunofluorescence technique, mentioned in Example 5B.

### Example 3

### Neo-organ Construction

Neo-organs were built with the genetically modified fibroblasts obtained in Example 2, and a biocompatible material made of paratetrafluoroethylene (or Gore-Tex™) fibers coated with types III and I collagen threads and basic fibroblast growth factor (bFGF). The lattice of this artificial structure retracted within a few days in culture medium and the neo-organs were then ready for implantation into the peritoneal cavity of humanized severe combined immunodeficient (SCID) mice.

### Example 4

### Implantation of Neo-organ in Immunodeficient Mice

Three groups of experiments were carried out: 2F5 (Group 2F5), sCD4-IgG (Group sCD4-IgG) or 2F5 plus sCD4-IgG (Group 2F5+sCD4-IgG) neo-organs were built and implanted into SCID mice as described below.

Ten SCID mice, controlled for agammaglobulinaemia were anaesthetized with phenobarbital. After median laparatomy, the neo-organs obtained in Example 3, maintained in bFGF-suplemented medium, were aseptically placed into the peritoneal cavity. These structures became strongly vascularized 1 or 2 weeks after their implantation in mice, as a result of the trophic and angiogenic properties of bFGF. SCID mice with neo-organs were bled weekly and checked for the presence of 2F5mAb or sCD4-IgG in serum samples. Three weeks after the neo-organ implantation, an optimal amount of recombinant molecules was reached.

At this time, 4×10⁷ of human CD4 cells (CEM T cell line) were injected intraperitoneally. Animals receiving CEM cells in the absence of previously implanted neo-organs were used as controls.

Two months after the implantation, SCID mice were killed to observe the structure and vascularization of the neo-organ.

### Example 5

### Detection of 2F5mAb and sCD4-IgG, in Vivo and ex vivo

A) Enzyme-linked immunosorbent Assay

Measures of 2F5 or sCD4-IgG in vitro and ex vivo were performed in cell culture supernatants and in plasma, respectively, using ELISA assays.

Briefly, microplates were coated by overnight incubation with purified ELDKWAS peptide (SEQ ID NO:2) or with gp160 proteins of the HIV-1 envelope. Inactivated plasma from neo-organ-implanted SCID mice from each group of Example 4, were then tested. 2F5mAb or sCD4-IgG were detected using horseradish peroxydase-conjugated goat anti-human IgG. The colored reaction was revealed using ortho-phenylenediamine as the substrate and stopped 3 minutes later by the addition of sulphuric acid 6 M. Optical densities were determined at 490 nm with the ELISA reader.

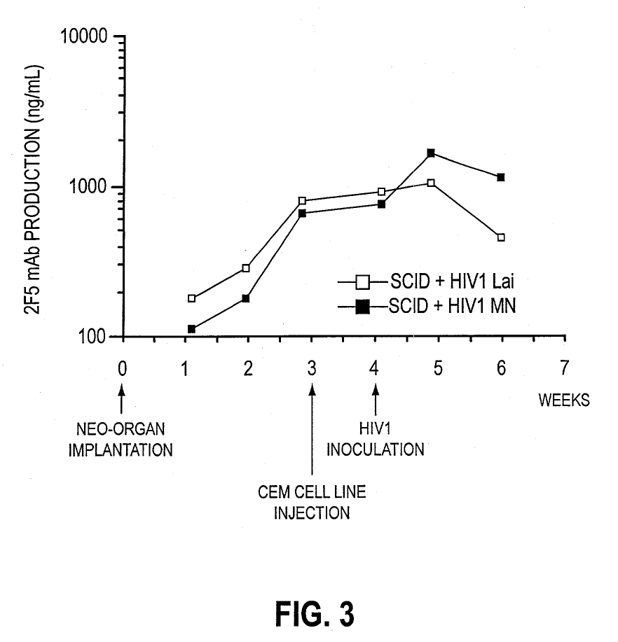

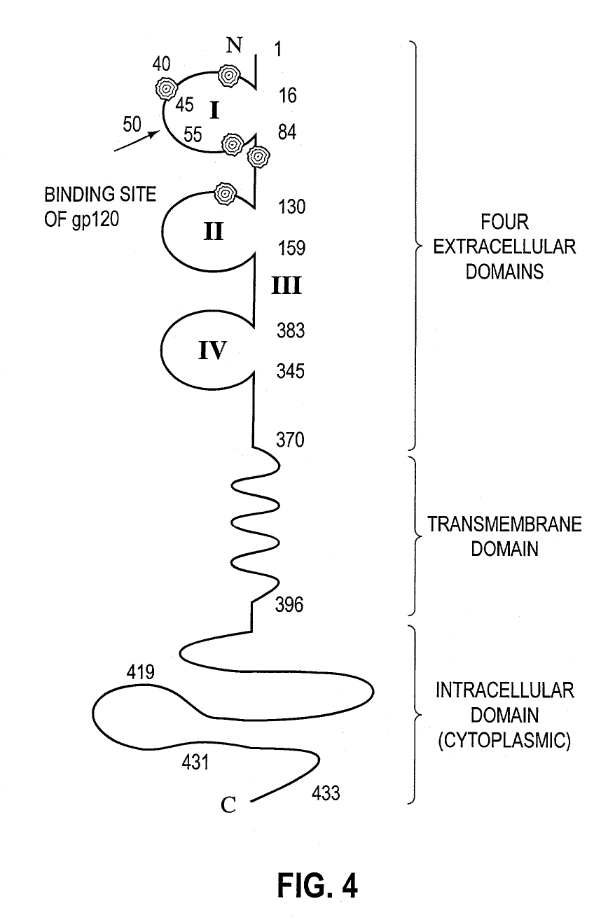

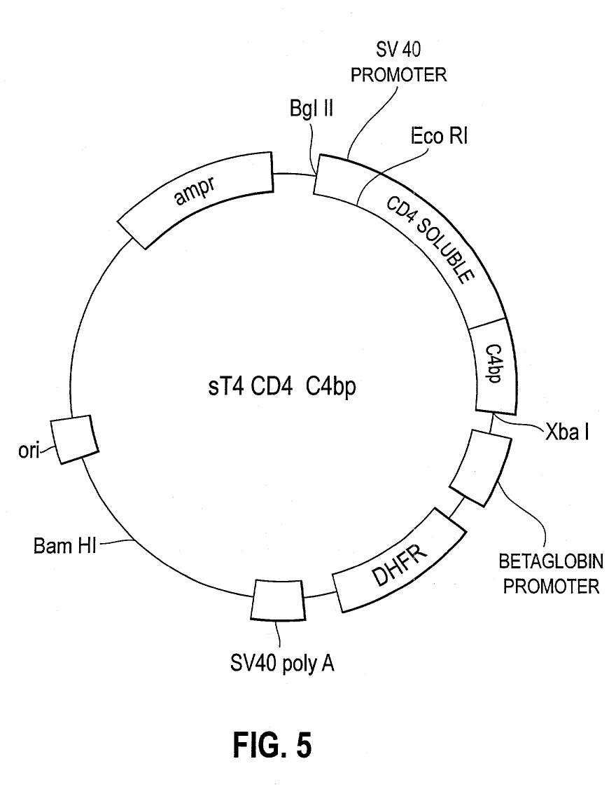

As evidenced on FIG. 3, in neo-organ implanted SCID mice, the follow-up of 2F5 production showed an increased secretion during the first 5 weeks after grafting. The mean plasma levels of 2F5 increased from 167 to 1000 ng/ml between weeks 1 and 5 in Lai-inoculated group (represented by □), and from 114 to 2000 ng/ml between the same weeks in MN-inoculated group (represented by ▪). At week 6, means of 450 and 1325 ng/ml 2F5 were found in the plasma of mice of the Lai and MN groups, respectively.

The inventors did not have any purified sCD4-IgG standard to quantify the production of the immunoadhesin in vitro and in vivo. Nevertheless, they were able to detect the molecules in cell supernatants and in mouse plasma by a qualitative ELISA assay as described above.

B) Immunofluorescence detection of 2F5 mAb

Adherent cells were fixed for 20 minutes in methanol acetone (v/v) before being treated. One step of incubation with a fluorescein-isothiocyanate-conjugated goat antibody directed against human IgG (heavy and light chains) diluted at 1:200 in phosphate-buffered saline ×1 with 5% fetal calf serum was performed. Ethidium bromide 1:100 in phosphate-buffered saline×1 was used to stain the nuclei.

### Example 6

### Detection of 2F5mAb and sCD4-IgG, in vivo

Measures of 2F5 and sCD4-IgG in vivo in [2F5+sCD4-IgG]-neo-organ-implanted SCID mice of Example 4 were performed using ELISA assays. These measures are carried out by a serum weekly assay.

A) Production of 2F5, in vivo

As already evidenced in Example SA and as shown in Table 1 below, the 2F5 concentration in plasma rapidly increases as from the first week after the neo-organ implantation. The mean plasma levels of 2F5 increased from 60 ng/ml to 1450 ng/ml between the first and the third weeks.

B) Production of sCD4-IgG in vivo

First, a qualitative evaluation of sCD4-IgG secretion was performed using ELISA assays. The mean plasma levels of sCD4-IgG increased from 10 ng/ml to 77 ng/ml between the first and the third weeks after the neo-organ implantation.

Second, a quantitative evaluation of sCD4-IgG secretion was performed using ELISA assays. As shown in Table 1 below, the mean plasma levels of sCD4-IgG increased from 4 ng/ml to 170 ng/ml between the first and the third weeks after the neo-organ implantation.

Table 1 also evidences that on the implantation day, neither 2F5, nor sCD4-IgG is secreted, in vivo.

TABLE 1

| | Secretion of transgenic molecules in plasma (ng/ml) | | | |
|---|---|---|---|---|
| Mice | Week 0 | Week 1 | Week 2 | Week 3 |
| 2F5 | | | | |
| SCID 1 | 0 | 120 | 360 | 710 |
| SCID 2 | 0 | 240 | 420 | 1075 |
| SCID 3 | 0 | 170 | 275 | 580 |
| SCID 4 | 0 | 60 | 160 | 615 |
| SCID 5 | 0 | 240 | 275 | 1450 |
| SCID 6 | 0 | 100 | 170 | 460 |
| SCID 7 | 0 | 160 | 225 | 1350 |
| SCD4-IgG | | | | |
| SCID 1 | 0 | 20 | 11 | 74 |
| SCID 2 | 0 | 4 | 16 | 50 |
| SCID 3 | 0 | 5 | 4 | 170 |
| SCID 4 | 0 | 19 | 10 | 34 |
| SCID 5 | 0 | 7 | 26 | 127 |
| SCID 6 | 0 | 12 | 21 | 46 |
| SCID 7 | 0 | 8 | 12 | 40 |

### Example 7

### HIV-1 Infection in vivo

10 A) Mice of Example 4

HIV-1 challenge in vivo

Four weeks after the intraperitoneal injection of CEM cells, SCID mice of Example 4 were challenged intravenously with 1000 TCID₅₀ (50% tissue-culture infectious dose) of HIV-1 (Lai or MN). The virus titration was carried out in CEM cell cultures, using the Nara technique (Nara et al.).

Measures of plasma and cellular viral loads, and reverse transcriptase activity

On one part of the cells removed from the spleen, the cellular proviral DNA was analyzed, using a quantitative-competitive polymerase chain reaction (PCR) The total DNA was extracted according to the trireagent method (Euromedex, Strasbourg, France) from HIV-1-infected and from non-infected CEM cells harvested from the spleens of killed SCID mice.

The plasma viral load was measured using the NASBA kit. A 140 base pair fragment of the gag gene was amplified from 2 μg DNA by quantitative-competitive PCR (HIV-1 PCR MIMIC Quantitation System kit; Clontech, Cambridge Bioscience, Cambridge, UK) using a SK 462/SK 431 primer pair in the presence of a 260 base pair heterologous fragment. A 10-fold dilution of the competitor (10⁶:1 copies) allowed the determination of the equimolariry of gag in each sample. The analysis of PCR products was performed by Southern blot hybridization using ³²P-labelled SK 102 and MIMIC probes (Clontech).

On the other part of the cells removed from the spleen, liver and tumor, cultures were performed to measure reverse transcriptase activity [Touraine JL, Sanhadji K. and Sembeil R. IMAJ, Gene therapy for HIV infection in the humanized SCID mouse model, in press].

B) Mice of Example 6

HIV-1 challenge in vivo

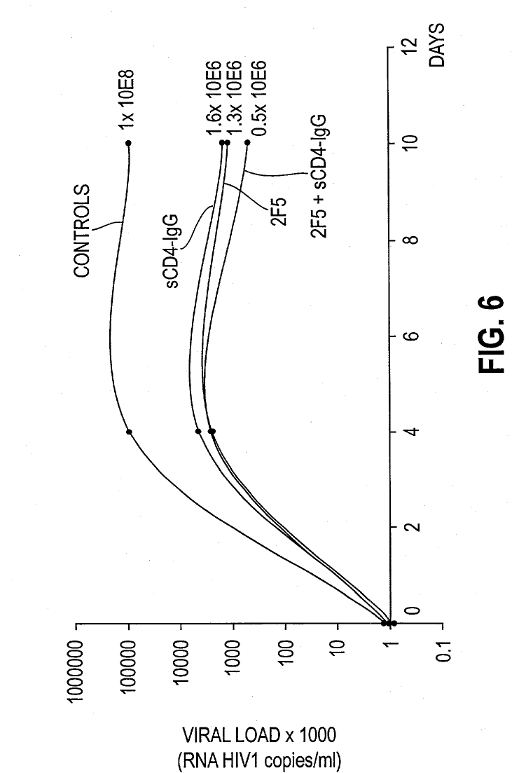

Three weeks after the intraperitoneal injection of CEM cells, SCID mice of Example 6 were challenged intravenously with 1000 TCID₅₀ (50% tissue-culture infectious dose) of HIV-1 (Lai). The plasma viral load is followed up by measure on day 0, 4 and 10 after HIV-1 inoculation. As illustrated on FIG. 6, the plasmatic viral load rapidly increases as from four days after the infection (up to 8 logarithms)

### Example 8

### In vivo Decrease of Viral Load Induced by 2F5 mAb

A) In vivo decrease of plasma viral load induced by 2F5mAb

As shown in Table 2, seven control SCID mice (four Lai and three MN) of Example 4 and six SCID mice with 2F5 mAb-producing neo-organs (four Lai and two MN) of Group 2F5 of Example 4 were tested for plasma viral load.

TABLE 2

| | HIV-1-RNA copies/ml at: | | | |
|---|---|---|---|---|
| Mice | Day 0 | Day 5 | Day 8 | Day 12 |

Controls

| | | | | |
|---|---|---|---|---|
| SCID 01 + Lai | <16 × 10³ | <16 × 10³ | 75 × 10⁷ | 2,9 × 10⁸ |
| SCID 02 + Lai | <16 × 10³ | <16 × 10³ | 66 × 10⁷ | 12 × 10⁸ |
| SCID 03 + Lai | ND | <8 × 10³ | 64 × 10⁷ | 9 × 10⁸ |
| SCID 04 + Lai | ND | <13 × 10³ | 94 × 10⁷ | 12 × 10⁸ |
| SCID 05 + Lai | ND | ND | ND | ND |
| SCID 5′ + MN | ND | <7 × 10³ | 31 × 10 | 13 × 10⁸ |
| SCID 6′ + MN | ND | <11 × 10³ | 9,5 × 10⁸ | 8,2 × 10⁸ |
| SCID 7′ + MN | ND | <3 × 10³ | 12 × 10⁸ | 12 × 10⁸ |
| 2F5 neo-organs | | | | |
| SCID 8 + Lai | ND | <27 × 10³ | ND | <2,5 × 10³ |
| SCID 9 + Lai | ND | <27 × 10³ | ND | <3 × 10³ |
| SCID 10 + Lai | ND | <18 × 10³ | ND | <5,5 × 10³ |
| SCID 11 + Lai | ND | <13 × 10³ | ND | <6 × 10 |
| SCID 12 + Lai | ND | ND | ND | ND |
| SCID 13 + Lai | ND | ND | ND | ND |
| SCID 14 + MN | ND | <25,5 × 10³ | ND | <16,5 × 10³ |
| SCID 15 = MN | ND | <4 × 10³ | ND | <4 × 10³ |

ND: Not done
Statistical analysis:
For Lai group: 2F5 neo-organs versus controls P < 0.0001
For MN group: 2F5 neo-organs versus controls P < 0.0002

In control animals, the plasma viral load increased significantly from day 5 to day 12 after HIV-1 challenge. The number of RNA copies reached 2.9-12×10⁸ and 8.2-13×10⁸ for Lai and MN isolates, respectively.

In mice implanted with 2F5-producing neo-organs, the plasma viral load was approximately 100 000-fold lower than that in control mice at day 12, both in the Lai and the MN groups. All tested implanted mice had a number of RNA copies below 16.5×10³ at day 12.

B) In vivo decrease of cellular viral load induced by 2F5mAb

At the time of death, quantitative detection of HIV-1 proviruses was performed by PCR in one part of the spleen cells recovered from control and from 2F5 neo-organ-implanted SCID mice inoculated with HIV-1. Proviral DNA was detected in all samples, but at different levels. It was approximately 100-fold lower in cells from mice with a 2F5 neo-organ than in cells from control mice. Whereas HIV-1 (Lai) experimental inoculation resulted in infection with either 3×10⁶ or 3×10⁵ HIV-1 copies in the five control mice, in SCID mice grafted with a 2F5 neo- organ, 3×10⁵ HIV-1 copies, 3×10⁴ HIV-1 copies or 3×10³ HIV-1 copies were detected. In mice injected with the MN strain, no statistically significant difference in the detection of intracellular HIV-1 provirus was observed between controls and 2F5-producer animals.

Cell cultures were performed using human T cells (CEM cells) recovered from the second part of various organs (spleen, tumor and liver) of grafted SCID mice. When CEM cells were removed from spleens, livers and tumors of mice implanted with 2F5 neo-organs, very low reverse transcriptase activity was consistently observed, in comparison with cultures obtained from HIV-infected control SCID.

### Example 9

### In vivo Decrease of Viral Load Induced by sCD4-IgG and 2F5mAb and sCD4-IgG

A) In vivo decrease of plasma viral load induced by sCD4-IgG

A separate experiment to compare 2F5mAb with sCD4-IgG showed that both compounds have comparable inhibitory effects on the plasma viral load of HIV Lai- or HIV MN-infected SCID-CEM mice as illustrated in Table 3. As a stock of Lai virus different from that in the previous experiment (Table 2) was used, the mean plasma viral load was higher. However it was significantly lower in all groups of implanted mice than in controls.

TABLE 3

| | HIV-1-RNA Copies/ml at: | | | |
|---|---|---|---|---|
| Mice | Day 0 | Day 4 | Day 10 | Day 15 |

Controls

| | | | | |
|---|---|---|---|---|
| SCID 01 + Lai | <1200 | 68 × 10⁶ | 100 × 10⁶ | 110 × 10⁶ |
| SCID 02 + Lai | <1100 | 96 × 10⁶ | 98 × 10⁶ | 160 × 10⁶ |
| SCID 03 + Lai | <870 | 100 × 10⁶ | 120 × 10⁶ | 100 × 10⁶ |
| SCID 04 + Lai | <1400 | 130 × 10⁶ | 95 × 10⁶ | 230 × 10⁶ |
| SCID 05 + Lai | <890 | 80 × 10⁶ | 110 × 10⁶ | 150 × 10⁶ |
| SCID 06 (− Lai) | <2000 | <1100 | <770 | <890 |
| SCID 07 (− Lai) | <1100 | <760 | <540 | <540 |
| SCID 08 (− Lai) | <640 | <1100 | <600 | <1100 |
| 2F5 neo-organs | | | | |
| SCID 09 + Lai | <1200 | 35 × 10⁵ | 15 × 10⁵ | 7,2 × 10⁵ |
| SCID 10 + Lai | <740 | <1100 | 25 × 10⁵ | 3,5 × 10⁵ |
| SCID 11 + Lai | <810 | 36 × 10⁵ | 8,1 × 10⁵ | 4,0 × 10⁵ |
| SCID 12 + Lai | <970 | <2000 | 11 × 10⁵ | 1,2 × 10⁵ |
| SCID 13 + Lai | <860 | 49 × 10⁵ | 19 × 10⁵ | 1,8 × 10⁵ |
| SCID 14 + Lai | <790 | 15 × 10⁵ | 15 × 10⁵ | 14 × 10⁵ |
| sCD4-IgG | | | | |
| neo-organs | | | | |
| SCID 15 + Lai | <750 | 27 × 10⁵ | 16 × 10⁵ | 6,4 × 10⁵ |
| SCID 16 + Lai | <900 | 38 × 10⁵ | 23 × 10⁵ | 8,7 × 10⁵ |
| SCID 17 + Lai | <1100 | 43 × 10⁵ | 23 × 10⁵ | 2,2 × 10⁵ |
| SCID 18 + Lai | <2600 | 57 × 10⁵ | 15 × 10⁵ | 2,7 × 10⁵ |
| SCID 19 + Lai | <800 | 64 × 10⁵ | 2,2 × 10⁵ | 9,1 × 10⁵ |
| SCID 20 + Lai | <860 | 0,25 × 10⁵ | 16 × 10⁵ | 2,6 × 10⁵ |
| 2F5 + sCD4-IgG | | | | |
| neo-organs | | | | |
| SCID 21 + Lai | <1100 | 28 × 10⁵ | 8,0 × 10⁵ | 6,7 × 10⁵ |
| SCID 22 + Lai | <1600 | 21 × 10⁵ | 5,1 × 10⁵ | 11 × 10⁵ |
| SCID 23 + Lai | <500 | 27 × 10⁵ | 5,0 × 10⁵ | 2,4 × 10⁵ |
| SCID 24 + Lai | <780 | 40 × 10⁵ | 4,8 × 10⁵ | 5,1 × 10⁵ |
| SCID 25 + Lai | <570 | 16 × 10⁵ | 8,3 × 10⁵ | 7,2 × 10⁵ |
| SCID 26 + Lai | <1400 | 27 × 10⁵ | 3,2 × 10⁵ | 1,1 × 10⁵ |
| SCID 27 + Lai | <1600 | 36 × 10⁵ | 7,7 × 10⁵ | 1,7 × 10⁵ |

Statistical analysis determined using a paired t-test
2F5 neo-organs versus controls P < 0.01.
sCD4-IgG neo-organs versus controls P < 0.01.
2F5 + sCD4-IgG neo-organs versus controls P < 0.01.
sCD4-IgG neo-organs versus 2F5 neo-organs not statistically significant

B) In vivo decrease of cellular viral load induced by sCD4-IgG

The effect of sCD4-IgG and 2F5 were therefore of the same order of magnitude.

At the time of death, the number of HIV-1 proviral DNA copies was low in the spleen cells of mice implanted with neo-organs secreting sCD4-IgG compared with controls (Table 4).

TABLE 4
Summary of HIV-1 proviral DNA detection, by
quantitative-competitive polymerase chain reaction, in
spleen cells of control, 2F5 or sCD4-IgG or 2F5 + sCD4-IgG
neo-organ-grafted, SCID mice, infected with HIV-1 (Lai).

| | | | | | | | |
|---|---|---|---|---|---|---|---|
| | 3 × 10⁵ | | 3 × 10⁴ | | 3 × 10³ | | 3 × 10² |
| Competitor copies | \| | \| | \| | \| | \| | \| | \| |

Controls

| | | |
|---|---|---|
| SCID 01 + Lai | x* | |
| SCID 02 + Lai | | x |
| SCID 03 + Lai | x | |

2F5 neo-organs

| | | | |
|---|---|---|---|
| SCID 09 + Lai | | x | |
| SCID 10 + Lai | | x | |
| SCID 11 + Lai | | | x |
| SCID 12 + Lai | x | | |
| SCID 13 + Lai | x | | |
| sCD4-IgG | | | |

| | | | |
|---|---|---|---|
| SCID 15 + Lai | | | x |
| SCID 16 + Lai | x | | |
| SCID 17 + Lai | | x | |

| | |
|---|---|
| SCID 18 + Lai | x |
| SCID 19 + Lai | x |
| 2F5 + sCD4-IgG | |

| | | |
|---|---|---|
| SCID 21 + Lai | | x |
| SCID 22 + Lai | x | |
| SCID 23 + Lai | | x |
| SCID 24 + Lai | | x |
| SCID 25 + Lai | | x |
| SCID 26 + Lai | | x |

*Crosses determine the equimolarity of the copy number of the HIV-1 gag fragment between the target DNA and the competitor DNA.

The Table shows that the efficacy of [2F5 plus sCD4-IgG] is better by a ten factor than any of 2F5 and sCD4-IgG, and that is efficacy is almost the same for all tested mice.

C) In vivo decrease of plasma viral load induced by both 2F5 and sCD4-IgG

When both molecules were produced together in vivo, again an important anti-HIV activity was demonstrable (Table 4).

D) In vivo decrease of cellular viral load induced by both 2F5 and sCD4-IgG

At the time of death, the number of HIV-1 proviral DNA copies was low in the spleen cells of mice implanted with neo-organs secreting 2F5 plus sCD4-IgG compared with controls (Table 5). In this group, a more homogeneous and potent effect was observed compared to neo-organ secreting only one of 2F5 and sCD4-IgG.

### Example 10

### Cooperation Between Secreted 2F5 and sCD4IgG

The production in vivo of 2F5 and sCD4 in SCID mice of Example 7B, decreases the viral load from 1,5 logarithm on day 4 to 2 logarithm on day 10, as shown on FIG. 6.

The effects of 2F5 and sCD4-IgG on the plasmatic viral load in vivo in SCID-Hu mice is summarized in Table 5 below.

TABLE 5

| | HIV-1-RNA copies/ml : | | |
|---|---|---|---|
| Mice | Day 0 | Day 4 | Day 10 |
| Controls HIV⁺ (5) | 1200 | 9.6 × 10⁷ | 1.0 × 10⁸ |
| 2F5 + HIV-1 (6) | 880 | 2.5 × 10⁶ | 1.3 × 10⁶ |
| SCD4-IgG + HIV-1 (6) | 880 | 4.5 × 10⁶ | 1.6 × 10⁶ |
| 2F5 + sCD4-IgG + HIV-1 (6) | 1100 | 2.7 × 10⁶ | 0.5 × 10⁶ |

The cooperation between 2F5 and sCD4-IgG thus secreted allows to reach about ten days after the HIV-1 injection a response three times higher than the one with either 2F5 or sCD4-IgG separately.

### REFERENCES

1. Ho D, Avidon UN, Perelson AS, Chen W, Leonard ) M, Markowitz M. Rapid turnover of plasma virions and CD4 lymphocytes in HIV-1 infection. Nature 1995, 373:123-126.

2. Ho D, Moudgil T, Alam M. Quantitation of human immunodeficiency virus type 1 in the blood of infected persons. N Engl J Med 1989, 321:1621-1625.

3. Saksela K, Stevens C, Rubinstein P, Baltimore D. Human immunodeficiency virus type 1 mRNA expression in peripheral blood cells predicts disease progression independently of the numbers of CD4+ lymphocytes. Proc Nail Acad Sci USA 1994, 91:1104-1108.

4. Pantaleo G, Graziosi C, Demarest J F, et al. HIV infection is active and progressive in lymphoid tissue during the clinically latent stage of disease. Nature 1993, 362:355-358.

5. Piatak M Jr, Saag M S, Yang L C, et al. High levels of HIV-1 in plasma during all stages of infection determined by competitive PCR. Science 1993, 259:1749-1754.

6. McCune I M, Namikawa R, Kaneshima H, Shultz L D, Lieberman M, Weissman IL. The SCID-hu mice: murine model for the analysis of the human hematolymphoid differentiation and function. Science 1988, 241:1632-1639.

7. Sanhadji K, Chargui J L, Touraine J L. Long-term chimera produced by transplantation of human fetal liver cells into severe combined immunodeficiency (SCID) mouse. Transplant Proc 1993, 25:452.

8. Namikawa R, Kaneshima H, Lieberman M, Weissman I L, McCune J M. Infection of the SCID-hu mice by HIV-1. Science 1988, 242:1684-1686.

9. Sanhadji K, Leissner P, Firouzi R, et al. Experimental gene therapy: the transfer of Tat-inducible interferon genes protects human cells against HIV-1 challenge in vitro and in vivo in severe combined immunodeficient mice. AIDS 1997, 11:977-986.

10. Karpas A, Hewlett I K, Hill F, et al. Polymerase chain reaction evidence for human immunodeficiency virus 1 neutralization by passive immunization in patients with AIDS and AIDS-related complex. Proc Natl Acad Sci USA 1990, 87:7613-7617.

11. Emini E, Schleif W A, Nunberg J H, et al. Prevention of HIV-1 infection in chimpanzees by gp120 V3 domain-specific monoclonal antibody. Nature 1992, 355:728-730.

13. Mascola J R, Lewis M G, Stiegler G, et al. Protection of macaques against pathogenic simian/human immunodeficiency virus 89.6PD by passive transfer of neutralizing antibodies. J Virol 1999, 73:4008-4018.

14. Pantaleo, G, Menzo S, Vaccarezza N, et al. Studies in subject with long-term nonprogressive human immunodeficiency virus infection. N Engl J Med 1995, 332:209-216.

15. Parren P W, Wang M, Trkola A, et al. Antibody neutralizationresistant primary isolates of human immunodeficiency virus type 1. J Virol 1998, 72:1270-1274.

16. Li A, Baba T W, Sodroski J, et al. Synergistic neutralization of a chimeric SIV/HIV type 1 virus with combinations of human antiHIV type 1 envelope monoclonal antibodies or hyperimmune globulins. AIDS Res Hum Retroviruses 1997, 13:647-656.

17. Mascola J R, Louder M K, VanCott T C, et al. Potent and synergistic neutralization of human immunodeficiency virus (HIV) type 1 primary isolates by hyperimmune anti-HIV immunoglobulin combined with monoclonal antibodies 2F5 and 2G12. J Virol 1997, 71:7198-7206.

18. Schenten D, Marcon L, Karlsson G B, et al. Effects of soluble CD4 on simian immunodeficiency virus infection of CD4-positive and CD4-negative cells. J Virol 1999, 73:5373-5380.

19. Klasse P J, Moore J P Quantitative model of antibody and soluble CD4-mediated neutralization of primary isolates and T-cell lineadapted strains of human immunodeficiency virus type 1. J Virol 1996, 70:3668-3677.

20. Trkola A, Ketas T, Kewalramani V N, et al. Neutralization sensitivity of human immunodeficiency virus type 1 primary isolates to antibodies and CD4-based reagents is independent of coreceptor usage. J Virol 1998, 72:1876-1885.

21. Schacker T, Collier A C, Coombs R, et al. Phase 1 study of highdose, intravenous rsCD4 in subjects with advanced HIV1 infection. J Acquir Immune Defic Syndr Hum Retrovirol 1995, 9:145-152.

22. Meng T C, Fischl M A, Cheeseman S H, et al. Combination therapy with recombinant human soluble CD4-immunoglobulin G and zidovudine in patients with HIV infection: a phase 1 study. J Acquir Immune Defic Syndr Hum Retrovirol 1995, 8:152-160.

23. Morgan R A, Ballet-Bitterlich G, Ragheb J A, Wong-Staal F, Gallo R C, Anderson W E Further evaluation of soluble CD4 as an antiHIV type 1 gene therapy: demonstration of protection of primary human peripheral blood lymphocytes from infection by HIV type 1. AIDS Res Hum Retroviruses 1994, 10:1507-1515.

24. Muster T, Steindl F, Purtscher M, et al. A conserved neutralizing epitope on gp41 of human immunodeficiency virus type 1. J Virol 1993, 67:6642-6647.

25. Klatzmann D R, McDougal J5, Maddon P J. The CD4 molecule and HIV infection. lmmunodefic Rev 1990, 2:43-66.

26. Moullier P, Bohl D, Heard J M, Danos O. Correction of lysosomal storage in the liver and spleen of MPS VII mice by implantation of genetically-modified skin fibroblasts. Nat Genet 1993, 4:154159.

27. Nara P L, Hatch W C, Dunlop N M, et al. Simple, rapid, quantitative, syncytium-forming microassay for detection of human immunodeficiency virus neutralizing antibody. AIDS Res Hum Retroviruses 1987, 3:283-302.

28. Buchacher A, Predl R, Strutzenberger K, et al. Generation of human monoclonal antibodies against HIV-1 proteins; electrofusion and EBV-transformation for PBLs immortalization, AIDS Research and Human Retroviruses 1994, 10:359-369.

29. Conley A J, Kessler II J A, Boots L J, et al. Neutralization of divergent human immunodeficiency virus type 1 variants and primary isolates by IAM-41-2F5, an anti-gp41 human monoclonal antibody. Proc Natl Acad Sci USA 1994, 91:3348-3352.

30. Muster T, Steindl F, Purtscher M, et al. A conserved neutralizing epitope on gp41 of human immunodeficiency virus type 1. I Virol 1993, 67:6642-6647.

31. Muster T, Guinea R, Trkola A, et al. Cross-neutralizing activity against divergent human immunodeficiency virus type 1 isolates induced by the gp41 sequence ELDKWAS. J Virol 1994, 68:4031-4034.

32. Li A, Katinger H, Posner M R, et al Synergistic neutralization of simian-human immunodeficiency virus SHIV-vpu+ by triple and quadruple combinations of human monoclonal antibodies and high-titer anti-human immunodeficiency virus type 1 immunoglobulins. I Virol 1998, 72:3235-3240.

33. Conley A l, Kessler II J A, Boots L J, et al. The consequence of passive administration of anti-human immunodeficiency virus type 1 neutralizing monoclonal antibody before challenge of chimpanzees with a primary virus isolate. J Virol 1996,70:6751-6758.

34. Parren P W H I, Ditzel H J, Gulizia R J, et a!. Protection against HIV1 infection in hu-PBL-SCID mice by passive immunization with a neutralizing human monoclonal antibody against the gp120 CD4-binding site. AIDS 1995, 9:1-6.

35. Andrus L, Prince A M, Bernal 1, et al. Passive immunization with a human immunodeficiency virus type 1 neutralizing monoclonal antibody in hu-PBL-SCID mice: isolation of a neutralization escape variant. J Infect Dis 1998, 177:889-897.

36. Buchacher A, Predl R, Tauer C, et al. Human monoclonal antibodies against gp41 and gp120 as potential agent for passive immunization. Vaccines 1992, 92:191-195.

37. Smith D H, Byrn R A, Marsters S A, Gregory T, Groopman J E, Capon D J. Blocking of HIV -1 infectivity by a soluble, secreted form of the CD4 antigen. Science 1987, 238:1704-1707.

38. Deen KC, McDougal J S, Inacker R, et al. A soluble form of CD4 (T4) protein inhibits AIDS virus infection. Nature 1988, 331:82-84.

39. Byrn R A, Sekigawa I, Chamow S M, et al. Characterization of in vitro inhibition of human immunodeficiency virus by purified recombinant CD4. J Virol 1989, 64:4370-4375.

40. Valere T, Bohl D, Klatzmann D, Danos O, Sonigo P, Heard JM. Continuous secretion of human soluble CD4 in mice transplanted with genetically modified cells. Gene Ther 1995, 2:197-202.

41. Capon D J, Chamow S M, Mordenti J, et al. Designing CD4 immunoadhesins for AIDS therapy. Nature 1989, 337:525-531.

42. Allaway G P, Davis-Bruno K L, Beaudry G A, et al. Expression and characterization of CD4-IgG2, a novel heterotetramer that neutralizes primary HIV-type 1 isolates. AIDS Res Hum Retroviruses 1995, 11, 533-539.

43. Tsubota H, Winkler G, Meade H M, Jakubowski A, Thomas DW Letvin N. CD4-Pseudomonas exotoxin conjugates delay but do not fully inhibit human immunodeficiency virus replication it lymphocytes in vitro. J Clin Invest 1990, 86:1684-1689.

44. Sullivan N, Sun Y, Sattentau Q, et al. CD4-induced conformational changes in the human immunodeficiency virus type 1 gp120 glycoprotein: consequences for virus entry and neutralization. J Virol 1998, 72:4694-4703.

45. Demaria S, Bushkin Y. Soluble CD4 induces the binding of human immunodeficiency virus type 1 to cells via the V3 loop of glycoprotein 120 and specific sites in glycoprotein 41. AIDS: Res Hum Retroviruses 1996, 12:281-290.

46. Sattentau Q, Zolla-Pazner S, Poignard F Epitope exposure or functional, oligomeric HIV1 gp41 molecules. Virology 1995, 206:713-717.

47. Ugolini S, Mondor I, Parren P W, et al. Inhibition of virus attachment to CD4+target cells is a major mechanism of T cell line-adapted HIV1 neutralization. J Exp Med 1997, 186:1287-1298.

48. Frankel S S, Steinman R M, Michael N L, et al. Neutralizing monoclonal antibodies block human immunodeficiency viru! type 1 infection of dendritic cells and transmission to T cell. J Viro! 1998, 72:9788-9794.

49. U.S. Pat. No. 5,817,767; WO-94/19017

50. Shinya E. et al., Biomed & Pharmacother 1999, 53:471-483

51. Barré-Sinoussi F, Cherman J C, Rey F, Nugeyre M T, Chamaret S, Gruest J, Dauguet C, Axler-Blin C, Brun-Vézinet F, Rouzioux C, Rozenbaum W and Montagnier L. Isolation of a T-lymphotropic retrovirus from a patient at risk of acquired immune deficiency syndrome, (AIDS). Science 1983, 220, 868-870

52. Gallo R C, Salahuddin S Z, Popovic M, Shearer G M, Kaplan M, Haynes B F, Palker T J and Markham P D. Frequent detection and isolation of cytopathic retroviruses (HTLV-III) from patients with AIDS and at risk for AIDS. Science 1984, 224, 500-503

53. Wain-Hobson S, Sonigo P, Danos O, Cole S & Alizon M. Nucleotide sequence of the AIDS virus, LAV. Cell 1985, 40, 9-17

54. Ratner L, Hasetine W, Patarca R, Livak K J, Starcich B, Josephs S F, Doran E R, Rafalski J A, Witehorn E A, Baumeister K, Ivanoff L, Petteway S R Jr, Pearson M L, Lautenberger J A, Papas T S, Ghrayeb J, Chang N T, Gallo R C and Wong-Staal F. Complete nucleotide sequence of the AIDS virus, HTLV-III. Nature 1985, 313, 277-284.

55. Klatzmann D, Champagne E, Chamaret S, Gruest J, Guetard D, Hercend T, Gluckman J C and Montagnier L. T-lymphocytes T4 molecule behaves as the receptor for human retrovirus LAV. Nature 1984, 312, 767-768.

56. H. Kattinger, 1992, Septiéme Colloque des Cent Gardes, 299-303.

57. EP-0 570 357

58. Wolf et al., Science 1990, 2, 1465-1468.

59. Robbins et al. 1998, Tibtech, 16,35-40.

60. Felgner et al., 1987, Proc. West. Pharmacol. Soc. 32, 115-121.

61. Hodgson and Solaiman, 1996, Nature Biotechnology 14, 339-342.

62. Remy et al., 1994, Bioconjugate Chemistry, 5, 647-654.

63. Lathe et al., 1987, Gene 57, 193-201.

64. Lupton and Levine, 1985, Mol. Cell. Biol. 5, 2533-2542.

65. Yates et al., Nature 313, 812-815.

66. Summers and Sherrat, 1984, Cell 36, 1097-1103.

67. Chinnasamy N. et al., Human Gene Therapy 2000, 11(13):1901-1909

68. Brown M D, Schätzlein A G, Uchegbu I F, Gene delivery with synthetic (non viral) carriers, Int. J. Pharm. 2001, 229, 1-21

69. Graham and Prevect, 1991, in Methods in Molecular Biology, Vol 7, p 109-128; Ed: E. J. Murey, The Human Press Inc.

70. Zakharchuk et al., Arch. Virol., 1993, 128: 171-176.

71. Spibey and Cavanagh, J. Gen. Virol. 1989, 70: 165-172.

72. Jouvenne et al., Gene, 1987, 60: 21-28.

73. Mittal et al., J. Gen. Virol., 1995, 76: 93-102.

74. McIvor et al., 1987, Mol. Cell Biol. 7, 838-848.

75. WO-96/08574

76. Tabin et al., 1982, Mol. Cell Biol. 2, 426-436) and SRα.

77. Takebe et al., 1988, Mol. Cell Biol. 8, 466-472.

78. Chen et al., 1995, J. Clin. Invest. 96, 2775-2782.

79. Schrewe et al., 1990, Mol. Cell. Biol. 10, 2738-2748.

80. Vile et al., 1993, Cancer Res. 53, 3860-3864.

81. Harris et al., 1994, Gene Therapy 1, 170-175.

82. Kanai et al., 1997, Cancer Res. 57, 461-465.

83. Sanhadji K., Grave L., Touraine J L;, Leissner P., Rouzioux C., Firouzi R., Kehrli L., Tardy J C and Mehtali M. Gene Transfer of anti-gp41 antibody and CD4 immunoadhesin strongly reduces the HIV-1 load in humanized severe combined immunodeficient mice, AIDS 2000, 14, 2813-2822.

84. Mc Ivor et al., 1987, Mol. Cell Biol. 7, 838-848

1. A composition comprising one or more nucleotide fragments, wherein the one or more nucleotide fragments taken together comprise at least (1) an nucleotide sequence (a) encoding human soluble CD4, (2) an nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and (3) the nucleic elements required for replicating each said nucleotide sequences (a) and (b), in a host cell, when said host cell divides and for expressing under control each of said nucleotide sequences (a) and (b) in said host cell.

2. The composition of claim 1, comprising an expression cassette comprising at least nucleotide sequence (a) and nucleotide sequence (b) and the nucleic elements required for expressing them under control in said host cell.

3. The composition of claim 1, comprising at least
a first expression cassette comprising nucleotide sequence (a), and the nucleic elements required for expressing it under control in said host cell, and
a second expression cassette comprising nucleotide sequence (b), and the nucleic elements required for expressing it under control in said host cell.

4. The composition of claim 1, comprising a vector comprising at least nucleotide sequence (a) and nucleotide sequence (b), and the nucleic elements required for expressing them under control in said host cell.

5. The composition of claim 1, comprising at least
a first recombinant vector comprising nucleotide sequence (a), and the nucleic elements required for expressing it under control in said host cell, and
a second recombinant vector comprising nucleotide sequence (b), and the nucleic elements required for expressing it under control in said host cell.

6. The composition of claim 1, wherein the nucleotide sequence (a) encodes sCD4 multimer.

7. The composition of claim 1, wherein the nucleotide sequence (a) further comprises a nucleotide sequence encoding a constant region of an immunoglobulin.

8. The composition of claim 7, wherein the constant region is a constant region of 2F5 monoclonal antibody.

9. The composition of claim 1, wherein nucleotide sequence (a) further comprises nucleotide sequences encoding the heavy chain and the light chain of 2F5 monoclonal antibody.

10. The composition of claim 4, wherein said vector is selected among the group consisting of adenoviral vectors, lentiviral vectors, second-generation adenoviral vectors, retroviral vectors, chimeric viral vectors and synthetic vectors.

11. The composition of claim 5, wherein at least one of said first and said second recombinant vectors is selected among the group consisting of adenoviral vectors, lentiviral vectors, second-generation adenoviral vectors, retroviral vectors, chimeric viral vectors and synthetic vectors.

12. The composition of claim 11, wherein at least one of said first and said second recombinant vectors is of murine Moloney leukemia retrovirus type.

13. The composition of claim 1, wherein said nucleic elements comprise a promoter.

14. The composition of claim 13, wherein the promoter is the promoter of mouse phosphoglycerate kinase type 1.

15. A host cell which comprises at least an expression cassette selected among the group consisting of an expression cassette of claim 2, the first expression cassette of claim 3 and the second expression cassette of claim 3.

16. A host cell which comprises at least a vector selected among the group consisting of a vector of claim 4, the first vector of claim 3 and the second vector of claim 3.

17. The host cell of claim 15, wherein said host cell is selected among the group consisting of fibroblasts, lymphocytes and stem cells.

18. The host cell of claim 16, wherein said host cell is selected among the group consisting of fibroblasts, lymphocytes and stem cells.

19. A tissue of genetically modified cells comprising a plurality of host cells of claim 17.

20. A tissue of genetically modified cells comprising a plurality of host cells of claim 18.

21. An implant of genetically modified cells comprising a plurality of host cells of claim 17.

22. An implant of genetically modified cells comprising a plurality of host cells of claim 18.

23. The implant of claim 21, wherein said host cells are selected among the group consisting of fibroblasts, lymphocytes, stem cells.

24. The implant of claim 22, wherein said host cells are selected among the group consisting of fibroblasts, lymphocytes, stem cells.

25. A method for treating an infectious disease, wherein a composition of claim 1, is administered by gene therapy, to a mammal or a patient.

26. The method of claim 25, wherein said infectious disease is caused by HIV-1 retrovirus.

27. A method for treating infectious disease, wherein a composition of claim 2, is administered to a mammal or a patient.

28. The method of claim 27, wherein said infectious disease is caused by HIV-1 retrovirus.

29. A method for treating infectious disease, wherein at least (1) a first expression cassette comprising a nucleotide sequence (a) encoding human soluble CD4 and nucleic elements required for replicating nucleotide sequence (a) in a host cell, when said host cell divides, and for expressing under control said nucleotide sequence (a) in said host cell, (2) a second expression cassette comprising a nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and nucleic elements required for replicating nucleotide sequence (b), when said host cell divides, and for expressing under control said nucleotide sequence (b) in said host cell,
are administered by gene therapy to a mammal or a patient.

30. The method of claim 29, wherein said first expression cassette and said second expression cassette are administered, concomitantly or separately.

31. The method of claim 29, wherein said infectious disease is caused by HIV-1 retrovirus.

32. A method for treating infectious disease, wherein at least (1) a first recombinant vector comprising a nucleotide sequence (a) encoding human soluble CD4 and nucleic elements required for replicating nucleotide sequence (a) in a host cell, when said host cell divides, and for expressing under control said nucleotide sequence (a) in said host cell, (2) a second recombinant vector comprising a nucleotide sequence (b) comprising at least nucleotide sequences encoding the heavy chain and the light chain of immunoglobulin IgG3, said IgG3 being directed against at least one of the peptide selected from the group consisting of SEQ ID NO:2 to SEQ ID NO:26, and nucleic elements required for replicating nucleotide sequence (b), when said host cell divides, and for expressing under control said nucleotide sequence (b) in said host cell,
are administered by gene therapy to a mammal or a patient.

33. The method of claim 32, wherein said first recombinant vector and said second recombinant vector are administered, concomitantly or separately.

34. The method of claim 32, wherein said infectious disease is caused by HIV-1 retrovirus.

35. A method for treating infectious disease, by gene therapy, wherein at a least one host cell of claim 15 is administrated by gene therapy to a mammal or a patient.

36. The method of claim 36, wherein said infectious disease is caused by HIV-1 retrovirus.

37. A method for treating infectious disease, wherein an implant of claim 21 is administrated by gene therapy, to a mammal or a patient.

38. The method of claim 37, wherein said infectious disease is caused by HIV-1 retrovirus.


SEQUENCE LISTING (33 sequences)

SEQ ID NO: 1 - DNA, 1745 bp, human sCD4
caagcccagaccctgccatttctgtgggctcaggtccctactgctcagcaagccccttcctccctcggcaaggccacaatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctatgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtgccaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttgaggcacgaggccaggcagatcccacttgcagcctccccaggtgtctgccccgcgtttcctgcctgcggaccagatgaatgtagcagatcccacgctctggcctcctgttcgtcctccctacaatttgccattgtttctcctgggttaggccccggcttcactggttgagtgttgctctctagtttccagaggcttaatcacaccgtcctccacgccatttccttttccttcaagcctagcccttctctcattatttctctctgaccctctccccactgctcatttggatcc

SEQ ID NO: 2 - protein, 6 aa, HIV-1 gp41 epitope
ELDKWA

SEQ ID NO: 3 - protein, 6 aa, HIV-1 gp41 epitope
ALDKWA

SEQ ID NO: 4 - protein, 6 aa, HIV-1 gp41 epitope
ELNKWA

SEQ ID NO: 5 - protein, 6 aa, HIV-1 gp41 epitope
ELDKWD

SEQ ID NO: 6 - protein, 6 aa, HIV-1 gp41 epitope
ALDTWA

SEQ ID NO: 7 - protein, 6 aa, HIV-1 gp41 epitope
QLDKWA

SEQ ID NO: 8 - protein, 6 aa, HIV-1 gp41 epitope
ELDTWA

SEQ ID NO: 9 - protein, 6 aa, HIV-1 gp41 epitope
GLDKWA

SEQ ID NO: 10 - protein, 6 aa, HIV-1 gp41 epitope
KLDEWA

SEQ ID NO: 11 - protein, 6 aa, HIV-1 gp41 epitope
SLDKWA

SEQ ID NO: 12 - protein, 6 aa, HIV-1 gp41 epitope
GRDKWA

SEQ ID NO: 13 - protein, 6 aa, HIV-1 gp41 epitope
GADKWA

SEQ ID NO: 14 - protein, 6 aa, HIV-1 gp41 epitope
AHEKWA

SEQ ID NO: 15 - protein, 6 aa, HIV-1 gp41 epitope
ACDQWA

SEQ ID NO: 16 - protein, 6 aa, HIV-1 gp41 epitope
GADKWG

SEQ ID NO: 17 - protein, 6 aa, HIV-1 gp41 epitope
GADKWN

SEQ ID NO: 18 - protein, 6 aa, HIV-1 gp41 epitope
GADKWC

SEQ ID NO: 19 - protein, 6 aa, HIV-1 gp41 epitope
GADKWV

SEQ ID NO: 20 - protein, 6 aa, HIV-1 gp41 epitope
GADKWH

SEQ ID NO: 21 - protein, 6 aa, HIV-1 gp41 epitope
GADKCH

SEQ ID NO: 22 - protein, 6 aa, HIV-1 gp41 epitope
GADKCQ

SEQ ID NO: 23 - protein, 6 aa, HIV-1 gp41 epitope
AYDKWS

SEQ ID NO: 24 - protein, 6 aa, HIV-1 gp41 epitope
AFDKWV

SEQ ID NO: 25 - protein, 6 aa, HIV-1 gp41 epitope
GPDKWG

SEQ ID NO: 26 - protein, 6 aa, HIV-1 gp41 epitope
ARDKWA

SEQ ID NO: 27 - DNA, 25 bp, Artificial sequence
  primer
ggaagcttccatggacatgagggtc

SEQ ID NO: 28 - DNA, 25 bp, Artificial sequence
  primer
aagaattcctaacactctcccctgt

SEQ ID NO: 29 - DNA, 25 bp, Artificial sequence
  primer
aaaagcttccatggagttgggtctg

SEQ ID NO: 30 - DNA, 25 bp, Artificial sequence
  primer
gggaattctcatttagccggagaca

SEQ ID NO: 31 - DNA, 573 bp, L, D1, J1, D2 domains of sCD4
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctatgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaag

SEQ ID NO: 32 - protein, 448 aa, human sCD4
MNRGVPFHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICVDQKEEVQLLVFGLTANSDTHLLQGQSLTLTLESPPGSSPSVQCRSPRKNIGGKTLSVSLELQDSGTWTCTVLQNKKVEFKIDIVVLAFKASSIVYKKEGEQVEFSFPLAFTVEKLTGSELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSKKTCQCPHRFQKTCSPI

SEQ ID NO: 33 - protein, 184 aa, L ,D1, J1, D2 domains of human sCD4
MNRGVPFHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICVDQKEEVQLLVFGLTANSDTHLLQGQSLTLTLESPPGSSPSVQCRSPRKNIGGKTLSVSLELQDSGTWTCTVLQNK